SEQUENCE LISTING (12 sequences)

SEQ ID NO: 1 - protein, 5 aa, Homo sapiens
DYAMH

SEQ ID NO: 2 - protein, 17 aa, Homo sapiens
VISYDGSNKYYADSVKG

SEQ ID NO: 3 - protein, 8 aa, Homo sapiens
DGLPAFDI

SEQ ID NO: 4 - protein, 11 aa, Homo sapiens
RASQSVRSNLA

SEQ ID NO: 5 - protein, 7 aa, Homo sapiens
DASSRAT

SEQ ID NO: 6 - protein, 9 aa, Homo sapiens
QQYGSSQLT

SEQ ID NO: 7 - protein, 117 aa, Homo sapiens
EVQLVQSGGGVVQPGGSLRLSCAASGFTFDDYAMHWVRQAPGKGLEWVAVISYDGSNKYYADSVKGRFTISRDNSKNTLYLQMNSLRAEDTAVYYCARDGLPAFDIWGKGTLVTVSS

SEQ ID NO: 8 - protein, 108 aa, Homo sapiens
EIVLTQSPATLSVSPGERAILSCRASQSVRSNLAWYQQKPGQPPRLVIYDASSRATGIPARFSGSGSGTDFTLTISSLEPEDFAAYYCQQYGSSQLTFGGGTKLEIKR

SEQ ID NO: 9 - protein, 240 aa, synthetic construct
EVQLVQSGGGVVQPGGSLRLSCAASGFTFDDYAMHWVRQAPGKGLEWVAVISYDGSNKYYADSVKGRFTISRDNSKNTLYLQMNSLRAEDTAVYYCARDGLPAFDIWGKGTLVTVSSGGGGSGGGGSGGGGSEIVLTQSPATLSVSPGERAILSCRASQSVRSNLAWYQQKPGQPPRLVIYDASSRATGIPARFSGSGSGTDFTLTISSLEPEDFAAYYCQQYGSSQLTFGGGTKLEIKR

SEQ ID NO: 10 - DNA, 720 bp, synthetic construct
  source 1..720: mol_type = other DNA
gaggtgcagctggtgcagtctgggggaggcgtggtacagcctggggggtccctgagactctcctgtgcagcctctggattcacctttgatgattatgccatgcactgggtccgtcaagctccagggaagggtctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagagatggcctccctgcttttgatatctggggcaaaggcaccctggtcaccgtctcgagtggtggaggcggttcaggcggaggtggttctggcggtggcggatcggaaattgtgttgacacagtctccagccaccctgtctgtgtctccaggggaaagagccatcctctcctgcagggccagtcagagtgttaggagcaacttagcctggtaccagcagaaacctggccagcctcccaggctcgtcatctatgatgcatccagcagggccactggcatcccagccaggttcagtggcagtgggtctgggacagacttcactctcaccatcagcagcctagagcctgaagattttgcagcgtattactgtcagcagtatggtagctcacagctcactttcggcggagggaccaagctggagatcaaacgt

SEQ ID NO: 11 - protein, 240 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 12 - protein, 243 aa, synthetic construct
QVQLQQSGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGARTTVTVSSGGGGSGGGGSGGGGSDIQAVVTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLTVL

(19) United States
(12) Patent Application Publication
(10) Pub. No.: US 2026/0022179 A1
(43) Pub. Date: Jan. 22, 2026

(54) ANTIBODY MONO-OR MULTI-SPECIFIC TO EPHRIN TYPE-A RECEPTOR 10, CHIMERIC ANTIGEN RECEPTOR T-CELL EXPRESSING THE SAME AND USES THEREOF

(71) Applicant: CHINA MEDICAL UNIVERSITY, Taichung (TW)

(72) Inventors: Long-Bin JENG, Taichung (TW); Shih-Ping LIU, Taichung (TW); Chie-Hong WANG, Taichung (TW)

(21) Appl. No.: 18/712,451

(22) PCT Filed: Nov. 22, 2022
(86) PCT No.: PCT/CN2022/133445

§ 371 Date: May 22, 2024

Related U.S. Application Data
(60) Provisional application No. 63/264,378, filed on Nov. 22, 2021.

(51) Int. Cl. C07K 16/28; A61K 39/00; A61K 47/68; A61K 51/10; A61P 35/00; G01N 33/68

(52) U.S. Cl. CPC C07K 16/2866; A61K 47/6803; A61K 47/6849; A61K 51/1033; A61K 51/1093; A61P 35/00; C07K 16/2809; C07K 16/283; G01N 33/6863; A61K 2039/505; A61K 2121/00; C07K 2317/53; C07K 2317/622; C07K 2317/73; C07K 2317/92; C07K 2319/02

(57) ABSTRACT

It relates to an antibody or antigen-binding fragment thereof that is mono-or multi-specific to ephrin type-A receptor 10 (EphA 10). It also relates to a pharmaceutical composition, a method for treating and/or preventing diseases and/or disorders caused by EphA 10 in a subject in need. and a method for detecting EphA 10 in a sample.

### PRIORITY INFORMATION

This application is a 371 National Phase Application of International Patent Application No. PCT/CN2022/133445, filed Nov. 22, 2022, which claims benefit of and priority to U.S. Provisional Patent Application No. 63/264,378, filed Nov. 22, 2021, the contents of which are incorporated by reference in their entirety.

### SEQUENCE LISTING

The instant application contains a Sequence Listing which is submitted electronically in .xml format and is hereby incorporated by reference in its entirety. The .xml copy, created on May 21, 2024, is named “G4590-14901NP_20240522_SeqList.xml” and is 11 kilobytes in size.

### FIELD OF THE DISCLOSURE

The present disclosure relates to an antibody or antigen-binding fragment thereof, which is specific to ephrin type-A receptor 10 (EphA10), a chimeric antigen receptor T-cell expressing the same and uses thereof.

### BACKGROUND OF THE DISCLOSURE

The utilization of therapeutic monoclonal antibodies for the treatment of human diseases including solid and hematological malignancies is well validated and established. Particularly useful for therapeutic applications are chimeric or humanized monoclonal antibodies due to their reduced immunological side effects. Among various immunotherapies developed for cancer treatments, chimeric antigen receptor (CAR) T-cell therapy is regarded as a powerful strategy. Generally, CAR T-cell therapy is performed by genetic modification of patient's T cells to express a tumor-specific CAR, ex vivo cell expansion and re-infusion back to the patient. Although an up to 92% full recovery rate is achieved in clinical trials in acute lymphocytic leukemia, the clinical experience of CAR T-cell therapy is still limited by several challenges, e.g., the expansion of CAR-T cells and the selection of tumor-specific antigen.

Thus, there is need for developing a novel approach to treating cancer.

### SUMMARY OF THE DISCLOSURE

The present disclosure provides a novel cancer antigen receptor and uses thereof.

Accordingly, the present disclosure provides an antibody or antigen-binding fragment thereof that is specific to an epitope of EphA10. The antibody according to the disclosure is thus useful for treating and/or preventing diseases and/or disorders caused by caused by or related to EphA10 activity and/or signaling. The antibody of the disclosure is also useful for detecting EphA10.

In some embodiments, the disclosure provides an antibody or antigen-binding fragment thereof that is specific to an epitope in EphA10; wherein the antibody or antigen-binding fragment thereof comprises complementarity determining regions (CDRs) of a heavy chain variable region and complementarity determining regions of a light chain variable region, wherein the complementarity determining regions of the heavy chain variable region comprise CDRH1, CDRH2 and CDRH3 regions, and the complementarity determining regions of the light chain variable region comprise CDRL1, CDRL2 and CDRL3 regions, and wherein:
the CDRH1 region comprises the amino acid sequence of SEQ ID NO: 1 or a substantially similar sequence thereof; the CDRH2 region comprises the amino acid sequence of SEQ ID NO: 2 or a substantially similar sequence thereof; the CDRH3 region comprises the amino acid of SEQ ID NO: 3 or a substantially similar sequence thereof; and
the CDRL1 region comprises the amino acid sequence of SEQ ID NO: 4 or a substantially similar sequence thereof; the CDRL2 region comprises the amino acid sequence of SEQ ID NO: 5 or a substantially similar sequence thereof; the CDRL3 region comprises the amino acid sequence of SEQ ID NO: 6 or a substantially similar sequence thereof.

In some embodiments, the antibody or antigen-binding fragment thereof comprises a heavy chain variable region comprising the amino acid sequence of SEQ ID NO: 7 or a substantially similar sequence thereof; and a light chain variable region comprising the amino acid sequence of SEQ ID NO: 8 or a substantially similar sequence thereof.

In some embodiments, the antibody is a monoclonal antibody, chimeric antibody, humanized antibody, human antibody, or nanobody.

In some embodiments, the disclosure provides a multi-specific antibody or antigen-binding fragment thereof comprising
a first antigen binding portion comprising the anti-EphA10 antibody or antigen-binding fragment thereof as disclosed herein; and
at least one second antigen binding portion.

In some embodiments, the second antigen binding portion is an anti-CD3 portion comprising an antibody or antigen-binding fragment thereof that is specific to an epitope in CD3 or an anti-CD16 portion comprising an antibody or antigen-binding fragment thereof that is specific to an epitope in CD16.

In some embodiments, the multi-specific antibody or antigen-binding fragment thereof comprises
the first antigen binding portion and the anti-CD3 portion;
the first antigen binding portion and the anti-CD16 portion; or
the first antigen binding portion, the anti-CD3 portion and the anti-CD16 portion.

In some embodiments, the multi-specific antibody or antigen-binding fragment thereof is in an arrangement of [the first antigen binding portion]-[the anti-CD3 portion], [the anti-CD3 portion]-[the first antigen binding portion], [the first antigen binding portion]-[the anti-CD16 portion], [the anti-CD16 portion]-[the first antigen binding portion], [the first antigen binding portion]-[the anti-CD3 portion]-[the anti-CD16 portion], [the first antigen binding portion]-[the anti-CD16 portion]-[the anti-CD3 portion], [the anti-CD3 portion]-[the first antigen binding portion]-[the anti-CD16 portion], [the anti-CD3 portion]-[the anti-CD16 portion]-[the first antigen binding portion], [the anti-CD16 portion]-[the anti-CD3 portion]-[the first antigen binding portion], or [the anti-CD16 portion]-[the first antigen binding portion]-[the anti-CD3 portion] from N-terminus to C-terminus.

In some embodiments, the multi-specific antibody or antigen-binding fragment thereof further comprises a linker located between any two of the first antigen binding portion and the second antigen binding portion.

In some embodiments, the multi-specific antibody or antigen-binding fragment thereof further comprises a secretion signal peptide.

In some embodiments, the multi-specific antibody or antigen-binding fragment thereof further comprises a protein purification tag.

In some embodiments of the disclosure, the antibody or antigen-binding fragment thereof or the multi-specific antibody or antigen-binding fragment thereof is conjugated with a therapeutic agent. Examples of the therapeutic agent include but are not limited to antimetabolites, alkylating agents, alkylating-like agents, DNA minor groove alkylating agents, anthracyclines, antibiotics, calicheamicins, antimitotic agents, topoisomerase inhibitors, HDAC inhibitor, proteasome inhibitors, and radioisotopes.

In some embodiments of the disclosure, the antibody or antigen-binding fragment thereof or the multi-specific antibody or antigen-binding fragment thereof is expressed on a surface of a cell. The cell may be an immune cell or a stem cell. In one embodiment of the disclosure, the immune cell is a T cell. In another aspect, the stem cell may be an induced pluripotent stem cell.

The present disclosure also provides a vector encoding the antibody or antigen-binding fragment thereof or the multi-specific antibody or antigen-binding fragment thereof.

In another aspect, the present disclosure provides a genetically engineered cell expressing the antibody or antigen-binding fragment thereof or the multi-specific antibody or antigen-binding fragment thereof, or containing the vector. The genetically engineered cell may be an immune cell or a stem cell. Also, the present disclosure provides an immune cell, which is differentiated from the genetically engineered cell.

The present disclosure provides a pharmaceutical composition comprising an effective amount of the antibody or antigen-binding fragment thereof, the multi-specific antibody or antigen-binding fragment thereof, the genetically engineered cell or the immune cell.

The present disclosure provides a method for inhibiting EphA10-mediated signaling in a subject in need, comprising administering to the subject the pharmaceutical composition. Alternatively, the present disclosure provides a pharmaceutical composition for use in inhibiting EphA10-mediated signaling in a subject in need, comprising an effective amount of the antibody or antigen-binding fragment thereof, the multi-specific antibody or antigen-binding fragment thereof, the genetically engineered cell or the immune cell as disclosed herein.

The present disclosure also provides a method for treating, prophylactic treating and/or preventing diseases and/or disorders caused by or related to EphA10 activity and/or signaling in a subject afflicted with the diseases and/or disorders, comprising administering to the subject the pharmaceutical composition. Alternatively, the present disclosure provides a pharmaceutical composition for use in treating, prophylactic treating and/or preventing diseases and/or disorders caused by or related to EphA10 activity and/or signaling in a subject afflicted with the diseases and/or disorders, comprising an effective amount of the antibody or antigen-binding fragment thereof, the multi-specific antibody or antigen-binding fragment thereof, the genetically engineered cell or the immune cell as disclosed herein.

The present disclosure still also provides a method for treating, prophylactic treating and/or preventing tumor in a subject afflicted with the tumor, comprising administering to the subject the pharmaceutical composition. In some embodiments of the disclosure, the tumor is a solid tumor. Examples of the tumor include but are not limited to renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, or melanoma. Alternatively, the present disclosure provides a pharmaceutical composition for use in treating, prophylactic treating and/or preventing tumor in a subject afflicted with the tumor, comprising an effective amount of the antibody or antigen-binding fragment thereof, the multi-specific antibody or antigen-binding fragment thereof, the genetically engineered cell or the immune cell as disclosed herein.

The present disclosure provides a method for detecting EphA10 in a sample comprising contacting the sample with the antibody or antigen-binding fragment thereof.

The present disclosure provides a method for neutralizing EphA10 in a subject in need, comprises administering to the subject the antibody or antigen-binding fragment thereof. Alternatively, the present disclosure provides a pharmaceutical composition for use in neutralizing EphA10 in a subject in need, comprising an effective amount of the antibody or antigen-binding fragment thereof, the multi-specific antibody or antigen-binding fragment thereof, the genetically engineered cell or the immune cell as disclosed herein.

The present disclosure provides a kit for detecting EphA10 in a sample, wherein the kit comprises the antibody or antigen-binding fragment thereof or the multi-specific antibody or antigen-binding fragment thereof.

The present disclosure is described in detail in the following sections. Other characteristics, purposes and advantages of the present disclosure can be found in the detailed description and claims.

### BRIEF DESCRIPTION OF THE DRAWINGS

EphAEphA FIG. 1 shows the results of binding specificity assay of the anti-EphA10 antibody CMU #5.

FIG. 2 shows the results of binding affinity assay of the anti-EphA10 antibody CMU #5.

FIG. 3 shows that cytotoxic effect of EphA 10 TriNTE on breast cancer cells.

FIG. 4 shows that cytotoxic effect of EphA10 BiTE on breast cancer cells.

FIG. 5 shows that cytotoxic effect of EphA10 BiKE on breast cancer cells.

FIG. 6 shows the effects of EphA10 TriNTE on T cell or NK cell activation.

FIG. 7 shows the effects of EphA10 TriNTE on cancer inhibition in an animal model.

FIG. 8 shows the result of immunostaining of co-culturing T cells, NK cells, BT-549 cells and EphA10 TriNTE.

FIG. 9 shows that the increased cytotoxic effect of anti-EphA10 CAR-T cells was validated by co-culturing the anti-EphA10 CAR T cells with breast cancer cells.

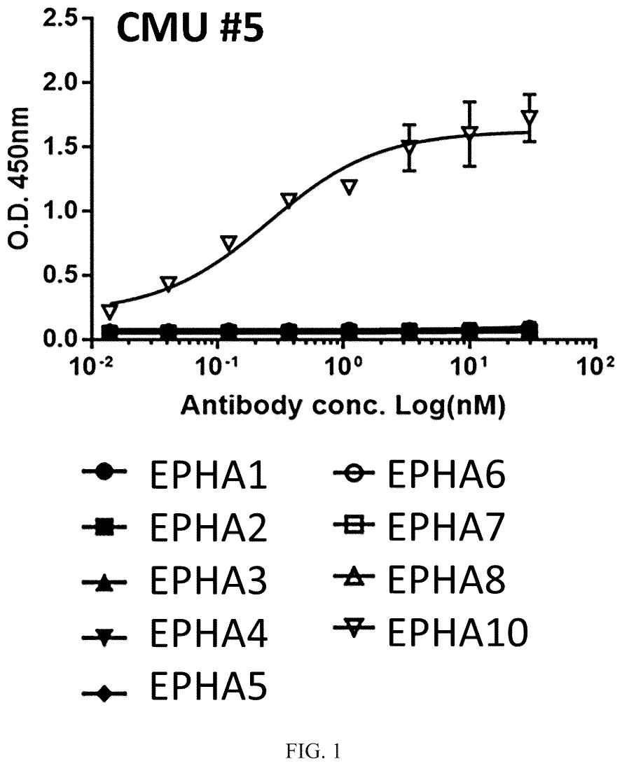

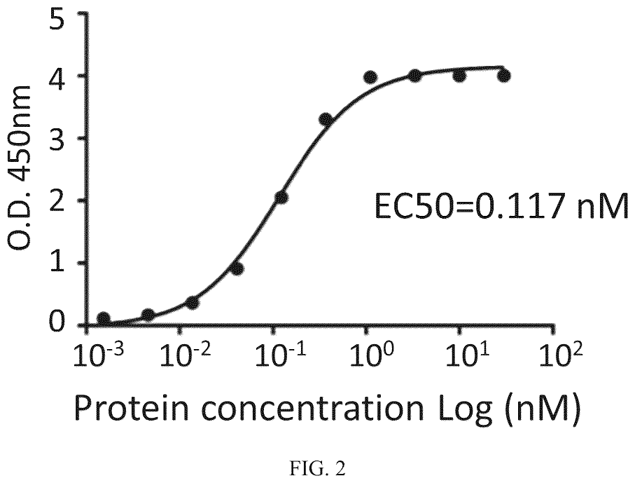

### DETAILED DESCRIPTION OF THE DISCLOSURE

It is understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise.

The term “antibody”, as used herein, means any antigen-binding molecule or molecular complex comprising at least one complementarity determining region (CDR) that specifically binds to or interacts with a particular antigen (EphA10). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H1}, C_{H2} and C_{H3}. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-EphA10 antibody (or antigen-binding portion thereof) may be identical to the human germline sequences, or may be naturally or artificially modified. An amino acid consensus sequence may be defined based on a side-by-side analysis of two or more CDRs.

As used herein, the term “being specific to” means that an antibody does not cross react to a significant extent with other epitopes.

As used herein, the term “epitope” refers to the site on the antigen to which an antibody binds.

As used herein, the term “complementarity determining region” (CDR) refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

The term “monoclonal antibody” as used herein is not limited to antibodies produced through hybridoma technology. A monoclonal antibody is derived from a single clone, including any eukaryotic, prokaryotic, or phage clone, by any means available or known in the art.

The term “chimeric” antibody as used herein refers to an antibody having variable sequences derived from a non-human immunoglobulin and human immunoglobulin constant regions, typically chosen from a human immunoglobulin template.

“Humanized” forms of non-human antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence.

As used herein, the term “nanobody” refers to an antibody comprising the small single variable domain (VHH of antibodies obtained from camelids and dromedarics. Antibody proteins obtained from members of the camel and dromedary (Camelus baclrianus and Calclus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex.

As used herein, “linker” or “spacer” peptide refers to short sequences of amino acids that join two polypeptide sequences (or nucleic acid encoding such an amino acid sequence). “Peptide linker” refers to the short sequence of amino acids joining the two polypeptide sequences. Exemplary of polypeptide linkers are linkers joining a peptide transduction domain to an antibody or linkers joining two antibody chains in a synthetic antibody fragment such as an scFv fragment. Linkers are well-known and any known linkers can be used in the provided methods. Exemplary of polypeptide linkers are (Gly-Ser)ₙ amino acid sequences, with some Glu or Lys residues dispersed throughout to increase solubility. Other exemplary linkers are described herein; any of these and other known linkers can be used with the provided compositions and methods.

As used in the present disclosure, the term “therapeutic agent” means any compound, substance, drug, drug or active ingredient having a therapeutic or pharmacological effect that is suitable for administration to a mammal, for example a human. As used herein, the term “immune cell” refers to cells that play a role in the immune response. Immune cells are of hematopoietic origin, and include lymphocytes, such as B cells and T cells; natural killer cells; myeloid cells, such as monocytes, macrophages, cosinophils, mast cells, basophils, and granulocytes.

As used herein, the term “T cell” includes CD4⁺ T cells and CD8⁺ T cells. The term T cell also includes T helper 1 type T cells, T helper 2 type T cells, T helper 17 type T cells and inhibitory T cells.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

The term “pluripotent” when used in reference to a “pluripotent cell” refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse teratoma formation assay. Pluripotency is also evidenced by the expression of embryonic stem (ES) cell markers, although a preferred test for pluripotency is the demonstration of the capacity to differentiate into cells of each of the three germ layers.

As used herein, the terms “iPS cell” and “induced pluripotent stem cell” are used interchangeably and refers to a pluripotent cell technically derived (e.g., induced by complete or partial reversal) from a differentiated cell (e.g. a non-pluripotent cell), typically an adult differentiated cell, for example, by contacting the cell with at least one compound of any compounds selected from xanthine, xanthosine, hypoxanthine, or analogs thereof, e.g. compounds with a xanthine nucleus.

As used herein, the term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “genetically engineered” or “genetic engineering” of cells means manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

In the context of cell ontogeny, the adjective “differentiated” or “differentiating” is a relative term. A “differentiated cell” is a cell that has progressed further down the developmental pathway than the cell it is being compared with. Thus, stem cells can differentiate to lineage-restricted precursor cells, which in turn can differentiate into other types of precursor cells further down the pathway, and then to an end-stage differentiated cell, which plays a characteristic role in a certain tissue type, and may or may not retain the capacity to proliferate further.

As used in the present invention, the term “pharmaceutical composition” means a mixture containing a therapeutic agent administered to a mammal, for example a human, for preventing, treating, or eliminating a particular disease or pathological condition that the mammal suffers.

As used herein, the term “therapeutically effective amount” or “efficacious amount” refers to the amount of an antibody that, when administered to a mammal or other subject for treating a disease, is sufficient to effect such treatment for the disease.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

“Cancer,” “tumor,” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

As used herein, the term “sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

The present disclosure develops an antibody or antigen-binding fragment thereof that is specific to an epitope in ephrin type-A receptor 10.

EphA 10 is a member of ephrin receptors, the largest subfamily of receptor tyrosine kinases (RTKs), and has an important function in development, angiogenesis, and cell differentiation. Previous studies indicated the therapeutic application of developing monoclonal antibody targeting EphA10 for cancer immunotherapy. It is found that (1) EphA10 levels are very low in normal tissues except the testis; (2) EphA10 levels are higher in various types of cancer than in normal tissues; (3) EphA10 deletion induced tumor regression by enhancing CTL-mediated antitumor immunity.

Particularly, the antibody or antigen-binding fragment thereof comprises complementarity determining regions (CDRs) of a heavy chain variable region and complementarity determining regions of a light chain variable region, wherein the complementarity determining regions of the heavy chain variable region comprise CDRH1, CDRH2 and CDRH3 regions, and the complementarity determining regions of the light chain variable region comprise CDRL1, CDRL2 and CDRL3 regions, and wherein:
the CDRH1 region comprises the amino acid sequence of SEQ ID NO: 1 or a substantially similar sequence having at least 90%, at least 95%, at least 98% or at least 99% sequence identity; the CDRH2 region comprises the amino acid sequence of SEQ ID NO: 2 or a substantially similar sequence having at least 90%, at least 95%, at least 98% or at least 99% sequence identity; the CDRH3 region comprises the amino acid sequence of SEQ ID NO: 3 or a substantially similar sequence having at least 90%, at least 95%, at least 98% or at least 99% sequence identity; and
the CDRL1 region comprises the amino acid sequence of SEQ ID NO: 4 or a substantially similar sequence having at least 90%, at least 95%, at least 98% or at least 99% sequence identity; the CDRL2 region comprises the amino acid sequence of SEQ ID NO: 5 or a substantially similar sequence having at least 90%, at least 95%, at least 98% or at least 99% sequence identity; the CDRL3 region comprises the amino acid sequence of SEQ ID NO: 6 or a substantially similar sequence having at least 90%, at least 95%, at least 98% or at least 99% sequence identity.

The sequence listing is shown in Table 1.

TABLE 1

| SEQ | | |
|---|---|---|
| ID | Sequence | |
| NO | name | sequence |
| 1 | VH CDR1 | DYAMH |
| 2 | VH CDR2 | VISYDGSNKYYADSVKG |
| 3 | VH CDR3 | DGLPAFDI |
| 4 | VL CDR1 | RASQSVRSNLA |
| 5 | VL CDR2 | DASSRAT |
| 6 | VL CDR3 | QQYGSSQLT |
| 7 | VH | EVQLVQSGGGVVQPGGSLRLSCAASGFTFDDYAMHWVRQAP |
| | | GKGLEWVAVISYDGSNKYYADSVKGRFTISRDNSKNTLYLQM |
| | | NSLRAEDTAVYYCARDGLPAFDIWGKGTLVTVSS |
| 8 | VL | EIVLTQSPATLSVSPGERAILSCRASQSVRSNLAWYQQKPGQPP |
| | | RLVIYDASSRATGIPARFSGSGSGTDFTLTISSLEPEDFAAYYCQ |
| | | QYGSSQLTFGGGTKLEIKR |
| 9 | scFv | EVQLVQSGGGVVQPGGSLRLSCAASGFTFDDYAMHWVRQAP |
| | amino acid | GKGLEWVAVISYDGSNKYYADSVKGRFTISRDNSKNTLYLQM |
| | | NSLRAEDTAVYYCARDGLPAFDIWGKGTLVTVSSGGGGSGGG |
| | | GSGGGGSEIVLTQSPATLSVSPGERAILSCRASQSVRSNLAWYQ |
| | | QKPGQPPRLVIYDASSRATGIPARFSGSGSGTDFTLTISSLEPEDF |
| | | AAYYCQQYGSSQLTFGGGTKLEIKR |
| 10 | scFv DNA | GAGGTGCAGCTGGTGCAGTCTGGGGGAGGCGTGGTACAGCC |
| | | TGGGGGGTCCCTGAGACTCTCCTGTGCAGCCTCTGGATTCAC |
| | | CTTTGATGATTATGCCATGCACTGGGTCCGTCAAGCTCCAGG |
| | | GAAGGGTCTGGAGTGGGTGGCAGTTATATCATATGATGGAA |
| | | GTAATAAATACTACGCAGACTCCGTGAAGGGCCGATTCACC |
| | | ATCTCCAGAGACAATTCCAAGAACACGCTGTATCTGCAAAT |
| | | GAACAGCCTGAGAGCTGAGGACACGGCTGTGTATTACTGTG |
| | | CGAGAGATGGCCTCCCTGCTTTTGATATCTGGGGCAAAGGC |
| | | ACCCTGGTCACCGTCTCGAGTGGTGGAGGCGGTTCAGGCGG |
| | | AGGTGGTTCTGGCGGTGGCGGATCGGAAATTGTGTTGACAC |
| | | AGTCTCCAGCCACCCTGTCTGTGTCTCCAGGGGAAAGAGCC |
| | | ATCCTCTCCTGCAGGGCCAGTCAGAGTGTTAGGAGCAACTT |
| | | AGCCTGGTACCAGCAGAAACCTGGCCAGCCTCCCAGGCTCG |
| | | TCATCTATGATGCATCCAGCAGGGCCACTGGCATCCCAGCC |
| | | AGGTTCAGTGGCAGTGGGTCTGGGACAGACTTCACTCTCAC |
| | | CATCAGCAGCCTAGAGCCTGAAGATTTTGCAGCGTATTACTG |
| | | TCAGCAGTATGGTAGCTCACAGCTCACTTTCGGCGGAGGGA |
| | | CCAAGCTGGAGATCAAACGT |
| 11 | CD3 scFv | QVQLQQSGAELARPGASVKMSCKASGYTFTRYTMHWVKQRP |
| | | GQGLEWIGYINPSRGYTNYNQKFKDKATLTTDKSSSTAYMQLS |
| | | SLTSEDSAVYYCARYYDDHYCLDYWGQGTTLTVSSGGGGSGG |
| | | GGSGGGGS |
| | | QIVLTQSPAIMSASPGEKVTMTCSASSSVSYMNWYQQKSGTSP |
| | | KRWIYDTSKLASGVPAHFRGSGSGTSYSLTISGMEAEDAATYY |
| | | CQQWSSNPFTFGSGTKLEIN. |
| 12 | CD16 scFv | QVQLQQSGAELVRPGTSVKISCKASGYTFTNYWLGWVKQRPG |
| | | HGLEWIGDIYPGGGYTNYNEKFKGKATVTADTSSRTAYVQVR |
| | | SLTSEDSAVYFCARSASWYFDVWGARTTVTVSS |
| | | GGGGSGGGGSGGGGS |
| | | DIQAVVTQESALTTSPGETVTLTCRSNTGTVTTSNYANWVQEK |
| | | PDHLFTGLIGHTNNRAPGVPARFSGSLIGDKAALTITGAQTEDE |
| | | AIYFCALWYNNHWVFGGGTKLTVL. |

In some embodiments, the antibody or antigen-binding fragment thereof comprises a heavy chain variable region comprising the amino acid sequence of SEQ ID NO: 7 or a substantially similar sequence thereof; and a light chain variable region comprising the amino acid sequence of SEQ ID NO: 8 or a substantially similar sequence thereof. In some further embodiments, the antibody or antigen-binding fragment thereof comprises a heavy chain variable region comprising the amino acid sequence of SEQ ID NO: 7 or a substantially similar sequence thereof having at least 90%, at least 95%, at least 98% or at least 99% sequence identity. In some further embodiments, the antibody or antigen-binding fragment thereof comprises a light chain variable region comprising the amino acid sequence of SEQ ID NO: 8 or a substantially similar sequence thereof having at least 90%, at least 95%, at least 98% or at least 99% sequence identity.

The antibody according to the disclosure can be full-length (for example, an IgG1 or IgG4 antibody) or may comprise only an antigen-binding portion (for example, a Fab, F(ab′)₂ or scFv fragment), and may be modified to affect functionality as needed.

Various techniques known to persons of ordinary skill in the art can be used to determine whether an antibody “is specific to one or more amino acids” within a polypeptide or protein. Exemplary techniques include, e.g., routine cross-blocking assay such as that described Antibodies, Harlow and Lane (Cold Spring Harbor Press, Cold Spring Harb., N.Y.), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody specifically binds is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. Sec, e.g., Ehring (1999) Analytical Biochemistry 267 (2):252-259; Engen and Smith (2001) Anal. Chem. 73:256A-265A.

One can easily determine whether an antibody is specific to the same epitope as, or competes for binding with, a reference anti-EphA10 antibody by using routine methods known in the art. For example, to determine if a test antibody binds to the same epitope as a reference anti-EphA 10 antibody of the disclosure, the reference antibody is allowed to bind to an EphA 10 protein. Next, the ability of a test antibody to bind to the EphA10 molecule is assessed. If the test antibody is able to bind to EphA10 following saturation binding with the reference anti-EphA10 antibody, it can be concluded that the test antibody binds to a different epitope than the reference anti-EphA10 antibody. On the other hand, if the test antibody is not able to bind to the EphA10 molecule following saturation binding with the reference anti-EphA10 antibody, then the test antibody may bind to the same epitope as the epitope bound by the reference anti-EphA10 antibody of the disclosure. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antibodies bind to the same (or overlapping) epitope if, e.g., a 1-, 5-, 10-, 20-or 100-fold excess of one antibody inhibits binding of the other by at least 50% but preferably 75%, 90% or even 99% as measured in a competitive binding assay. Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

The antibody also includes an antigen-binding fragment of a full antibody molecule. An antigen-binding fragment of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of an antigen-binding fragment includes: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody typically comprises at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (v) V_{H}-C_{H1}-CH₂-CH₃, (vi) V_{H}-CH₂-C_{H3}; (vii) V_{H}-C_{L}; (viii) V_{L}-C_{H1}; (ix) V_{L}-C_{H2}; (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The anti-EphA10 antibody disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes an antibody, and an antigen-binding fragment thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another mammalian germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes an anti-EphA10 antibody comprising variants of any of the V_{H}, V_{L}, and/or CDR amino acid sequences disclosed herein having one or more conservative substitutions. For example, the present disclosure includes an anti-EphA10 antibody having V_{H}, V_{L}, and/or CDR amino acid sequences with, e.g., 10 or fewer, 8 or fewer, 6 or fewer, 4 or fewer, etc. conservative amino acid substitutions relative to any of the V_{H}, V_{L}, and/or CDR amino acid sequences disclosed herein.

In some embodiments of the disclosure, the antibody according to the disclosure is a humanized antibody. In order to improve the binding affinity of the humanized antibody according to the disclosure, some amino acid residues in the human framework region are replaced by the corresponding amino acid residues in the species of CDRs; e.g. a rodent.

The antibodies of the present disclosure may be monospecific, bi-specific, or multispecific. Multispecific antibodies may be specific for different epitopes of one target polypeptide or may contain antigen-binding domains specific for more than one target polypeptide. The anti-EphA10 antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present disclosure includes bi-specific antibodies wherein one arm of an immunoglobulin is specific for EphA10 or a fragment thereof, and the other arm of the immunoglobulin is specific for a second therapeutic target or is conjugated to a therapeutic moiety.

In some embodiments, the disclosure provides a multi-specific antibody or antigen-binding fragment thereof comprising
a first antigen binding portion comprising the anti-EphA10 antibody or antigen-binding fragment thereof as disclosed herein; and
at least one second antigen binding portion.

In some embodiments, the second antigen binding portion is an anti-CD3 portion comprising an antibody or antigen-binding fragment thereof that is specific to an epitope in CD3 or an anti-CD16 portion comprising an antibody or antigen-binding fragment thereof that is specific to an epitope in CD16.

The term “cluster of differentiation 3” or “CD3,” as used herein, refers to any native CD3 from any vertebrate source, including mammals such as primates (e.g. humans) and rodents (e.g., mice and rats), unless otherwise indicated, including, for example, CD3ε, CD3γCD3α, and CD3β chains, and encompasses full-length, “unprocessed” CD3 (e.g., unprocessed or unmodified CD38 or CD3γ), as well as any form of CD3 that results from processing in the cell, such as a “processed” CD3γ) polypeptide without all or a portion of its signal peptide. CD3 is a multimeric protein complex, known historically as the T3 complex, and is composed of four distinct polypeptide chains; ε, γ, δ, and ζ, that assemble and function as three pairs of dimers (εγ, εδ, ζζ). The CD3 complex serves as a T cell co-receptor that associates noncovalently with the T cell receptor.

In one embodiment of the disclosure, the anti-CD3 portion is a scFv having sequence of SEQ ID NO: 11.

CD16 (also known as FcγRIII), a low affinity receptor for the Fc portion of some IgGs known to be involved in antibody-dependent cellular cytotoxicity (ADCC), is the best-characterized membrane receptor responsible for triggering of target cell lysis by NK cells (Mandelboim et al., 1999, PNAS 96:5640-5644). The majority (approximately 90%) of human NK cells express CD56 at low density (CD56^{dim}) and FcγRIII (CD16) at a high level (Cooper et al., 2001, Trends Immunol. 22:633-640). Human FcγRIII exists as two isoforms, FcγRIIIA and FcγRIIIB, that share 96% sequence identity in their extracellular immunoglobulin-binding regions (van de Winkel and Capel, 1993, Immunol. Today 14(5):215-221).

In some embodiment of the disclosure, the anti-CD16 portion is a scFv having sequence of SEQ ID NO: 12.

In some embodiments, the multi-specific antibody or antigen-binding fragment thereof comprises
the first antigen binding portion and the anti-CD3 portion;
the first antigen binding portion and the anti-CD16 portion; or
the first antigen binding portion, the anti-CD3 portion and the anti-CD16 portion.

In some embodiments, the multi-specific antibody or antigen-binding fragment thereof is in an arrangement of [the first antigen binding portion]-[the anti-CD3 portion], [the anti-CD3 portion]-[the first antigen binding portion], [the first antigen binding portion]-[the anti-CD16 portion], [the anti-CD16 portion]-[the first antigen binding portion], [the first antigen binding portion]-[the anti-CD3 portion]-[the anti-CD16 portion], [the first antigen binding portion]-[the anti-CD16 portion]-[the anti-CD3 portion], [the anti-CD3 portion]-[the first antigen binding portion]-[the anti-CD16 portion], [the anti-CD3 portion]-[the anti-CD16 portion]-[the first antigen binding portion], [the anti-CD 16 portion]-[the anti-CD3 portion]-[the first antigen binding portion], or [the anti-CD16 portion]-[the first antigen binding portion]-[the anti-CD3 portion] from N-terminus to C-terminus.

In some embodiments, the multi-specific antibody or antigen-binding fragment thereof further comprises a linker located between any two of the first antigen binding portion and the second antigen binding portion.

In some embodiments of the disclosure, the multi-specific antibody or antigen-binding fragment thereof further comprises a secretion signal peptide. The signal peptide (sometimes referred to as signal sequence, targeting signal, localization signal, localization sequence, transit peptide, leader sequence or leader peptide) as used herein refers to a short peptide located at the N-terminus of a protein that is destined towards the secretory pathway.

In some embodiment of the disclosure, the multi-specific antibody or antigen-binding fragment thereof further comprises a protein purification tag, such as 6×His purification tag (HHHHHH), a Myc-detection tag (EQKLISEEDL) and a strepII affinity purification tag (WSHPQFEK). This allows selection for the conjugated product, if Ni-NTA affinity resins (for the 6×His-tag) or streptactin affinity resins (for the strep II-tag) are employed to separate non-conjugated substrate from conjugated product.

The fusion protein according to the disclosure recognizes EphA 10 on tumor cells, CD3 on T-cells and/or CD16 on NK-cells (TriNTE, BITE, and BIKE). In some embodiments of the disclosure, the fusion protein is a T-cell engaging tri-specific construct that exists as three single-chain variable fragments (scFv) connected by a short linker. One scFv binds T-cells via the CD3 receptor; one scFv binds the tumor cell via a tumor-associated antigen and/or the other binds to NK-cells CD16 receptor.

While not wishing to be limited by theory, it is believed that the fusion protein according to the disclosure bring T cells and/or NK cells to cancer cell periphery. It achieves better effect than T cells or NK cells alone. In some embodiments of the disclosure, in the presence of human T-cells, TriNTE, BITE, and BiKE leads to the specific killing of human cancer cells.

In one embodiment of the disclosure, the antibody or antigen-binding fragment thereof or the multi-specific antibody or antigen-binding fragment thereof is conjugated with a therapeutic agent.

In some embodiments of the disclosure, the therapeutic agent represents a cytostatic or cytotoxic agent or an isotope-chelating agent with corresponding radioisotopes. Examples of the cytostatic or cytotoxic agent include, without limitation, antimetabolites (e.g., fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, gemcitabine, capecitibine, azathioprine, cytosine methotrexate, trimethoprim, pyrimethamine, or pemetrexed); alkylating agents (e.g., cmelphalan, chlorambucil, busulfan, thiotepa, ifosfamide, carmustine, lomustine, semustine, streptozocin, dacarbazine, mitomycin C, cyclophosphamide, mechlorethamine, uramustine, dibromomannitol, tetranitrate, procarbazine, altretamine, mitozolomide, or temozolomide); alkylating-like agents (e.g., cisplatin, carboplatin, nedaplatin, oxaliplatin, satraplatin, or triplatin); DNA minor groove alkylating agents (e.g., duocarmycins such as CC-1065, and any analogs or derivatives thereof; pyrrolobenzodiazapenes, or any analogs or derivatives thereof); anthracyclines (e.g., daunorubicin, doxorubicin, epirubicin, idarubicin, or valrubicin); antibiotics (e.g., dactinomycin, bleomycin, mithramycin, anthramycin, streptozotocin, gramicidin D, mitomycins (e.g., mitomycin C); calicheamicins; antimitotic agents (including, e.g., maytansinoids (such as DM1, DM3, and DM4), auristatins (including, e.g., monomethyl auristatin E (MMAE) and monomethyl auristatin F (MMAF)), dolastatins, cryptophycins, vinca alkaloids (e.g., vincristine, vinblastine, vindesine, vinorelbine), taxanes (e.g., paclitaxel, docetaxel, or a novel taxane), tubulysins, and colchicines); topoisomerase inhibitors (e.g., irinotecan, topotecan, camptothecin, etoposide, teniposide, amsacrine, or mitoxantrone); HDAC inhibitor (e.g., vorinostat, romidepsin, chidamide, panobinostat, or belinostat); proteasome inhibitors (e.g., peptidyl boronic acids); as well as radioisotopes such as At²¹¹, I¹³¹, I¹²⁵, Y⁹⁰, Re¹⁸⁶, Re¹⁸⁸, Sm¹⁵³, Bi²¹² or ²¹³, P³² and radioactive isotopes of Lu including Lu¹⁷⁷. Examples of the isotope-chelating agents include, without limitation, ethylenediaminetetraacetic acid (EDTA), diethylenetriamine-N,N,N′,N″,N″-pentaacetate (DTPA), 1,4,7,10-tetraazacyclododecane-N,N′,N″,N″′-tetraacetate (DOTA), 1,4,7,10-tetrakis (2-hydroxypropyl)-1,4,7,10-tetraazacyclododecane (THP), triethylenetetraamine-N,N,N′,N″,N″′,N″′-hexaacetate (TTHA), 1,4,7,10-tetraazacyclododecane-N,N′,N″,N″′-tetrakis (methylenephosphonate) (DOTP), and mercaptoacetyltriglycine (MAG3).

In one embodiment of the disclosure, the antibody or antigen-binding fragment thereof or the multi-specific antibody or antigen-binding fragment thereof is expressed on the surface of a cell. Particularly, the cell is a T-cell or a stem cell, such as an iPSC. Induced pluripotent stem cells can be reprogrammed by inducing Yamanaka factors into somatic cells. Being like embryonic stem cells, iPSCs have the ability to be differentiated into cells of three-germ layers without the concern of ethic issue. With this property, iPSCs exhibit promising applications for clinical use.

In some embodiments of the disclosure, the antibody or antigen-binding fragment thereof or the multi-specific antibody or antigen-binding fragment thereof is in a form of chimeric antigen receptor.

The term “chimeric antigen receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. The generation and construction of CAR are generalized by Jayaraman et al., EBioMedicine 58 (2020) 102931; Zhang et al., Biomarker Research (2017) 5:22; Feins et al., Am J Hematol. (2019) 94:S3-S9; and Roselli et al., J Clin Invest. 2021;131(2):c142030.

The antibody or antigen-binding fragment thereof or the multi-specific antibody or antigen-binding fragment thereof may be encoded in a vector encoding the antibody or antigen-binding fragment thereof. An exemplary vector is a lentivirus vector. “Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

In another aspect, the present disclosure provides a genetically engineered cell expressing the antibody or antigen-binding fragment thereof or the multi-specific antibody or antigen-binding fragment thereof or containing the vector. The genetically engineered cell may be an immune cell or a stem cell. Also, the present disclosure provides an immune cell, which is differentiated from the genetically engineered cell.

In one embodiment of the disclosure, anti-EphA10 iPSCs and T cells with lentivirus carrying EphA10 gene are generated. Furthermore, the iPSCs are differentiated these into CAR-immune cells. The cytotoxic effects of these CAR-immune cells on cancer cells are observed. The present disclosure provides an approach to overcome the specificity of tumor antigen and produce limitless CAR-immune cells for clinical cancer treatment application.

The disclosure provides pharmaceutical compositions comprising the antibody or antigen-binding fragment thereof or the multi-specific antibody or antigen-binding fragment thereof, genetically engineered cell or immune cell of the present disclosure. The pharmaceutical compositions of the disclosure are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient may vary depending upon the age and the size of the patient, target disease, conditions, route of administration, and the like. The preferred dose is typically calculated according to body weight or body surface area. When an antibody of the present disclosure is used for treating a condition or disease associated with EphA10 in an adult patient, it may be advantageous to intravenously administer the antibody of the present disclosure. Depending on the severity of the condition, the frequency and the duration of the treatment can be adjusted. Effective dosages and schedules for administering the antibody may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (sec, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The present disclosure provides a method for inhibiting EphA10-mediated signaling in a subject in need, comprising administering to the subject the pharmaceutical composition. Alternatively, the present disclosure provides a pharmaceutical composition for use in inhibiting EphA 10-mediated signaling in a subject in need, comprising an effective amount of the antibody or antigen-binding fragment thereof, the multi-specific antibody or antigen-binding fragment thereof, the genetically engineered cell or the immune cell as disclosed herein.

The present disclosure also provides a method for treating, prophylactic treating and/or preventing diseases and/or disorders caused by or related to EphA 10 activity and/or signaling in a subject afflicted with the diseases and/or disorders, comprising administering to the subject the pharmaceutical composition. Alternatively, the present disclosure provides a pharmaceutical composition for use in treating, prophylactic treating and/or preventing diseases and/or disorders caused by or related to EphA10 activity and/or signaling in a subject afflicted with the diseases and/or disorders, comprising an effective amount of the antibody or antigen-binding fragment thereof, the multi-specific antibody or antigen-binding fragment thereof the genetically engineered cell or the immune cell as disclosed herein.

The present disclosure still also provides a method for treating, prophylactic treating and/or preventing tumor in a subject afflicted with the tumor, comprising administering to the subject the pharmaceutical composition. In some embodiments of the disclosure, the tumor is a solid tumor. Examples of the tumor include but are not limited to renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, or melanoma. Alternatively, the present disclosure provides a pharmaceutical composition for use in treating, prophylactic treating and/or preventing tumor in a subject afflicted with the tumor, comprising an effective amount of the antibody or antigen-binding fragment thereof, the multi-specific antibody or antigen-binding fragment thereof the genetically engineered cell or the immune cell as disclosed herein.

The present disclosure provides a method for detecting EphA10 in a sample comprising contacting the sample with the antibody or antigen-binding fragment thereof.

The present disclosure provides a method for neutralizing EphA10 in a subject in need, comprises administering to the subject the antibody or antigen-binding fragment thereof. Alternatively, the present disclosure provides a pharmaceutical composition for use in neutralizing EphA10 in a subject in need, comprising an effective amount of the antibody or antigen-binding fragment thereof, the multi-specific antibody or antigen-binding fragment thereof the genetically engineered cell or the immune cell as disclosed herein.

The present disclosure provides a kit for detecting EphA10 in a sample, wherein the kit comprises the antibody or antigen-binding fragment thereof or the multi-specific antibody or antigen-binding fragment thereof or the multi-specific antibody or antigen-binding fragment thereof.

The anti-EphA10 antibody of the present disclosure may also be used to detect and/or measure EphA10, or EphA10-expressing cells in a sample, e.g., for diagnostic purposes. For example, an anti-EphA10 antibody, or fragment thereof, may be used to diagnose a condition or disease characterized by aberrant expression (e.g., over-expression, under-expression, lack of expression, etc.) of EphA10. Exemplary diagnostic assays for EphA10 may comprise, e.g., contacting a sample, obtained from a patient, with an anti-EphA10 antibody of the disclosure, wherein the anti-EphA10 antibody is labeled with a detectable label or reporter molecule. Alternatively, an unlabeled anti-EphA10 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure EphA10 in a sample include enzyme-linked immunosorbent assay (ELISA), radioimmunoassay (RIA), and fluorescence-activated cell sorting (FACS).

The following examples are provided to aid those skilled in the art in practicing the present disclosure.

### EXAMPLES

### Phage Display Library Screen

A human B cell-derived scFv phage display library (Creative BioLabs, USA) was used to pan against the recombinant human EphA10 extracellular domain protein (Glu34-Ala565), transcript variant 3, fused with DDK/His tag at C-terminal (Creative Biomart, USA, Catalog no. EPHA10-369H). Positive clones were screened using ELISA. A unique CMU #5 were identified by DNA sequencing. The binding specificity of the unique clones was confirmed by FACS using full-length human EphA 10-expressing NIH 3T3 cells.

### Construction of EphA10-scFv-CD3z-scFv-CD16-scFv, EphA10-scFv-CD3z-scFv, and EphA10-scFv-CD16-scFv Plasmid

Three plasmids were designed and constructed to generate TriNTE (EphA10 (CMU #5)-scFv (SEQ ID NO: 9)-CD3z-scFv (SEQ ID NO: 11)-CD16-scFv (SEQ ID NO: 12)), BITE (EphA10 (CMU #5)-scFv (SEQ ID NO: 9)-CD3z-scFv (SEQ ID NO: 11)), and BIKE (EphA10 (CMU #5)-scFv (SEQ ID NO: 9)-CD3z-scFv (SEQ ID NO: 12)). After assembly of the amino acid sequences for each antibody, the scFv sequence was synthesized and cloned into a eukaryotic expression vector.

### Anti-EphA10/anti-CD3/anti-CD16 TriNTE Antibody Generation

TriNTE, BITE, and BIKE were produced by transient transfection of the expression plasmids into HEK293 cells using the ExpiFectamine 293 Transfection Kit (Thermo Fisher Scientific). Briefly, plasmids were diluted in Opti-MEM, mixed with pre-diluted ExpiFectamine for 20-30 min at room temperature and added to HEK293 cells. Transfection efficiency was optimized to determine the best ratio of plasmids for TriNTE, BiTE, and BiKE production with good yield and purity. The supernatant from transfected cells was collected and filtered through a 0.45-μm filter unit 4-5 days after transfection (Nalgene). TriNTE, BITE, and BIKE in the supernatant were purified using anti-His beads. Purified TriNTE, BITE, and BiKE were then aliquoted and stored at −80° C.

### Enzyme-Linked Immunosorbent Assay (ELISA)

ScFv sequences were fused with mouse IgG2a sequence and sub-cloned into pcDNA3.1 plasmid. Three ScFv-mouse IgG2a plasmids were transfected into HEK293 cells. Three ScFv-mouse IgG2a antibodies were purified by protein A resin. Three antibodies were tested for indirect ELISA against coated 9 EPHA antigens (EphA1-8 and 10, R&D systems).

The results are shown in FIG. 1 and Table 2. CMU #5 is shown to only bind to EphA10 without binding to other EphAs with EC50 against EphA 10 measured by ELISA of 0.117 nM.

TABLE 2

| | CMU#5 (nM) | | | | | | | | | |
|---|---|---|---|---|---|---|---|---|---|---|
| | 30.0000 | 10.0000 | 3.3333 | 1.1111 | 0.3704 | 0.1235 | 0.0412 | 0.0137 | 0.0046 | 0.0015 |
| EphA10 | 4 | 4 | 4 | 4 | 3.277 | 2.057 | 0.928 | 0.375 | 0.17 | 0.104 |
| | 4 | 4 | 4 | 3.963 | 3.206 | 2.034 | 0.893 | 0.358 | 0.166 | 0.133 |
| | 4 | 4 | 4 | 3.968 | 3.426 | 2.063 | 0.912 | 0.366 | 0.175 | 0.109 |

### Cytotoxicity Assay

Human breast cancer cells (BT-549) were labeled with CFSE and incubated with purified human T and NK cells at the indicated E:T ratios in the presence of TriNTE, BiTE, BiTE, or PBS for 4 h. To monitor spontaneous death, CFSE-labeled target cells were cultured alone under identical conditions. After 4 h, cells were harvested and 7-aminoactinomycin D (7AAD) was added before analysis. Samples were thoroughly mixed and analyzed by flow cytometry.

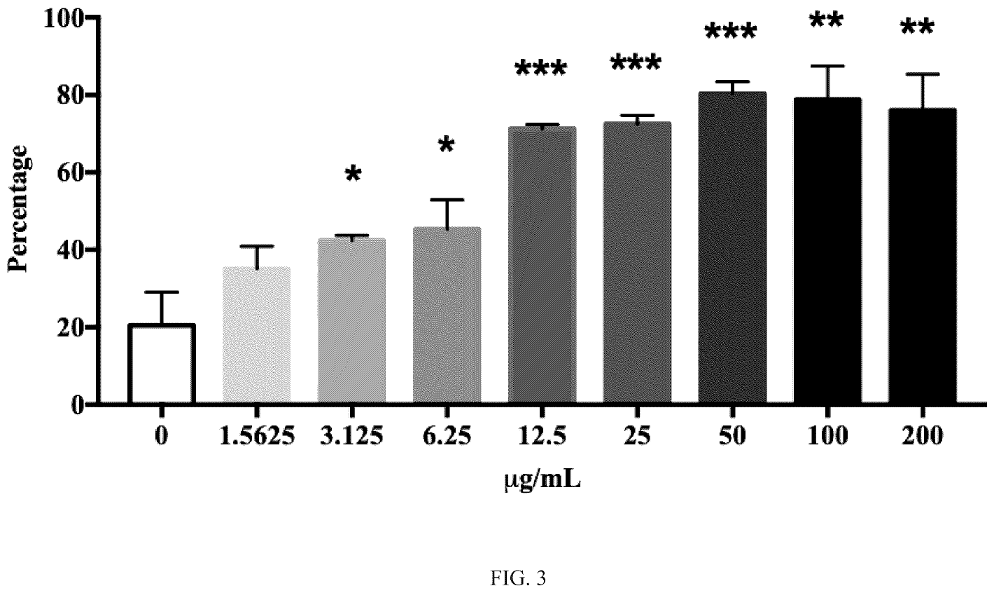

The results of EphA10 TriNTE are shown in FIG. 3. At more than 3.125 μg/ml, the cytotoxic effect was significantly higher than that of the control group. At more than 50 μg/ml, the cytotoxicity percentage was about 80%. We conclude that EphA10 TriNTE provides an effective anti-tumor activity.

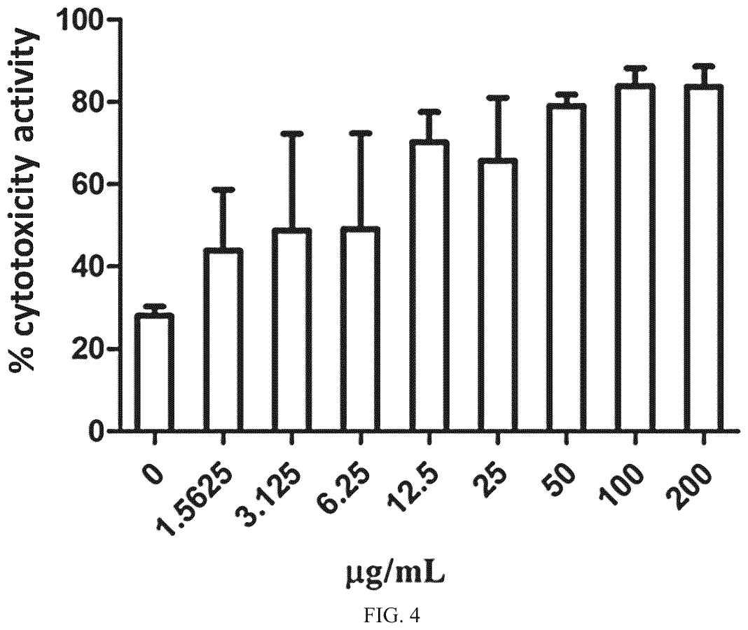

The results of EphA10 BiTE are shown in FIG. 4. At more than 50 μg/ml, the cytotoxicity percentage was about 80%. We conclude that EphA10 BiTE provides an effective anti-tumor activity.

The results of EphA10 BiKE are shown in FIG. 4. At more than 50 μg/ml, the cytotoxicity percentage was about 80%. We conclude that EphA10 BiKE provides an effective anti-tumor activity.

### Isolation and Purification of NK Cells and T-Cells

Redirected cellular cytotoxicity was examined with flow cytometry using human PBMCs or isolated T-cells and NK cells as effector cells and various EphA 10-positive human breast cancer cell lines. PBMCs were isolated from healthy donors with Ficoll density gradient centrifugation using standard procedures. Briefly, after centrifugation, cells were incubated in 10 ml erythrocyte lysis buffer (Sigma-Aldrich) at room temperature for 10 min, washed with PBS, resuspended in RPMI-1640 medium, at a concentration of 6×10⁶ cells/ml. Alternatively, T-cells were isolated from PBMCs using the Pan T-cell Isolation Kit II (Miltenyi Biotec). For T-cell activation, 2×10⁶ T-cells were cultured in 24-well plates coated with 1 μg anti-CD3 antibody and 1 μg anti-CD28 antibody (eBioscience). Cells were incubated for 3 days at 37° C. in a 5% CO₂ humidified incubator. NK cells were isolated from PBMCs using the NK Cell Isolation Kit (Miltenyi Biotec). Cells were washed once in FACS buffer (PBS, 2% FBS, and 0.02% NaN₃) and incubated at 4° C. in 200 μl for 30 min. Propidium iodide (PI) was added to a final concentration of 1 μg/ml, and samples were analyzed by flow cytometry (BD Biosciences). Target cell lysis was determined as the percentage of PI-stained cells. All experiments were performed in duplicates.

### Characterization of T-Cell Activation

T-cell activation was determined according to CD25 expression by flow cytometry analysis. Lymphocytes were incubated in 96-well plates with or without autologous target MDMs (E:T ratio of 4:1 and 8:1), and treated with TriNTE, BITE, and BIKE. Experiments were conducted in 50% ascites fluid. After 4 days of co-culture, lymphocytes were harvested and stained with anti-CD4, anti-CD8, anti-CD25, anti-CD69, anti-CD107a, and anti-HLA-DR antibodies.

### Characterization of NK Cell Activation

NK cell activation against tumor targets in the presence or absence of treatments was measured according to CD107a-mediated degranulation and IFN-y production using flow cytometry. Lymphocytes were incubated in 96-well plates with or without autologous target MDMs (E:T ratio of 4:1 and 8:1), and treated with TriNTE, BiTE, and BIKE. Experiments were conducted in 50% ascites fluid. After 4 days of co-culture, lymphocytes were harvested and stained with anti-CD69, anti-CD107a, anti-Trial, anti-IFN-γ, and anti-TNF-α antibodies.

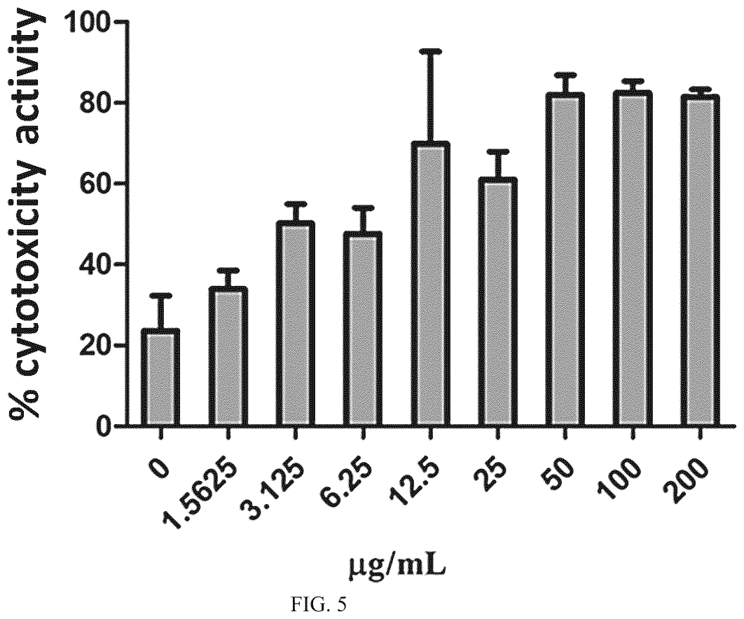

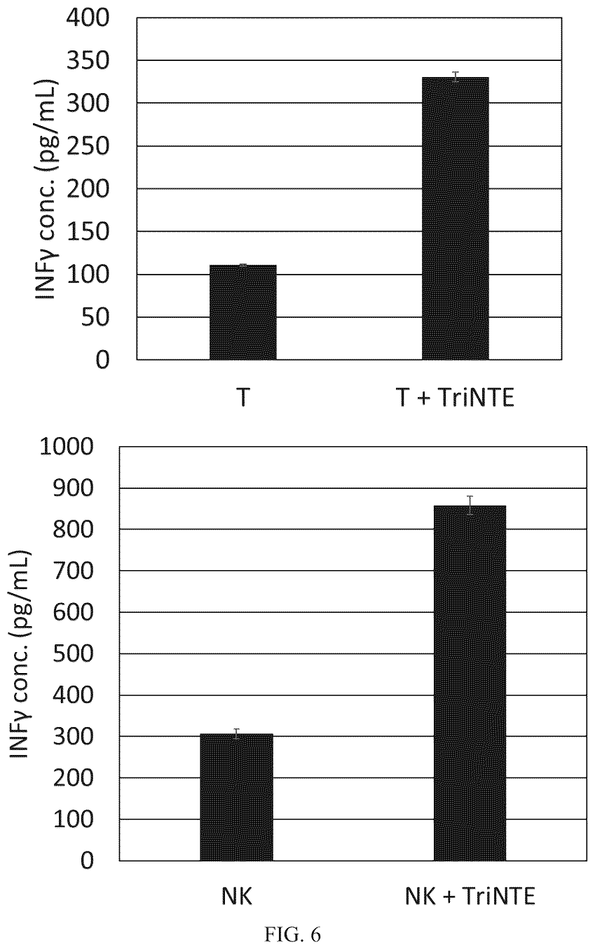

The results of K cell and NK cell activation are shown in FIG. 5. When co-culturing breast cancer cells and T or NK cells, the group treated with EphA10 TriNTE increased the release of cytokine by about three times. EphA10 TriNTE indeed increases the activation of T or NK cells and increases the release of cytokine.

### In Vivo Engraftment With Breast Cancer Cells

The in vivo efficacy of EphA10 TriNTE was evaluated in NSG mice that received a subcutaneous engraftment of 1×10⁶ MDA-MB-231 cells with 1×10⁶ non-stimulated PBMCs. Six animals per group were treated intravenously with 1 or 10 μg TriNTE, and control saline was administered on study days 0, 1, 2, and 3. Tumor growth in two perpendicular directions was measured on the indicated days with calipers, and tumor volume (mm3) was calculated using the following formula: V=(width2× length)/2 (Taki et al., 2015). IVIS will also be used to measure tumor growth in NSG mice.

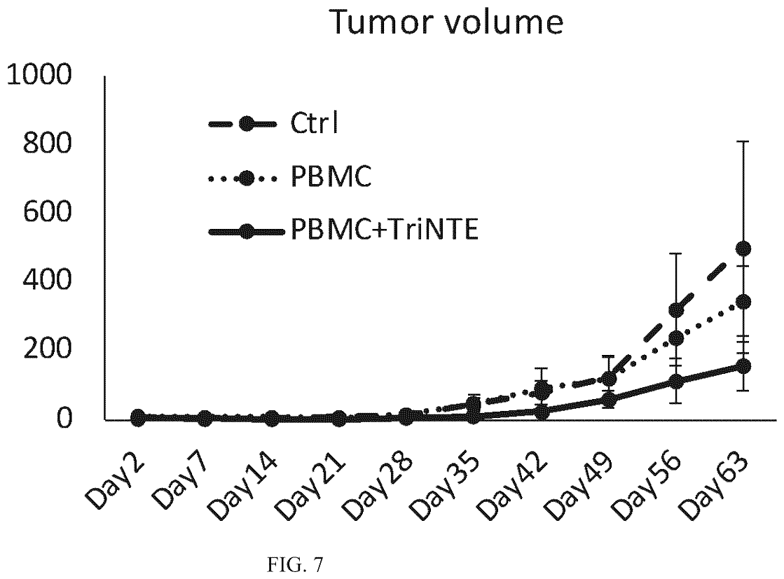

The results are shown in FIG. 7. The EphA10 TriNTE-treated group shows significant inhibition of tumor growth. Thus, EphA10 TriNTE shows in vivo effectiveness against EphA10-positive breast cancer tumors.

### Immunostaining

The T cells, NK cells and BT-549 cells were cultured with EphA10 TriNTE. Cells were fixed for 10 min with 4% PFA/PBS and incubated overnight at 4° C. with monoclonal antibodies. The next day, colonies were stained with DAPI for monitoring BT-549, with anti-CD3 (FITC) antibody and with anti-CD16 (TxRd) antibody.

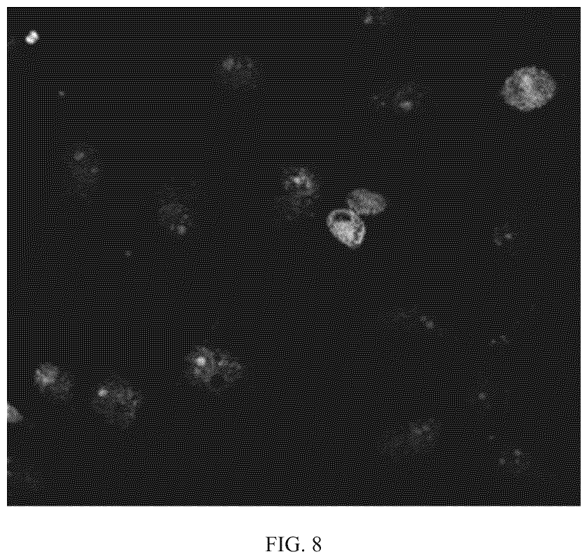

The results are shown in FIG. 8. EphA10 TriNTE binds to T cells, NK cells and BT-549 cells simultaneously.

### Construction of EphA10-CAR-T Lentiviral Vector

CMU #5 ScFv (SEQ ID NO: 9) was cloned and inserted to a third-generation CAR-T lentiviral vector containing CD8 hinge, CD28 TM and IC, 41-BB and CD35. The sequence of CMU #5 ScFv was synthesized and sub-cloned into a third-generation CAR-T lentiviral vector (pCDH-EFla-MCS) (Creative BioLabs, USA). To generate EphA10 CAR lentivirus, the third-generation CAR-T lentiviral vector and packaging vectors (LentiArt™ Virus Packaging Kit, Creative Biolabs, USA, Cat. No. CART-027CL) were co-transfected into HEK293 cells.

### Generation of EphA10-CAR T (EphA10-CAR T)

Human CD8⁺ T cells were used for the generation of EphA10-CAR T. Briefly, CD8⁺ T cells were stably transduced with CAR lentivirus encoding a CMU #5 ScFv (SEQ ID NO: 9). All EphA10-CAR T cell lines were maintained in KBM502 medium (Kohjin Bio, Japan). EphA10-CAR T cell lines were tested for mycoplasma contamination every 2 months.

### Cytotoxicity Assays

Cytotoxic potential of EphA10-CAR-T cells was evaluated in non-radioactive cytotoxicity assay. The EphA10-CAR-T cells were co-cultured with EphA10-positive MDA-MB-231 cells at an effector to target ratio of 10 (E/T ratio=10; effector cell: EphA10-CAR-T cell; target cell: MDA-MB-231 cell) for 24 hours. The cell viability of MDA-MB-231 was assayed with MTT assay.

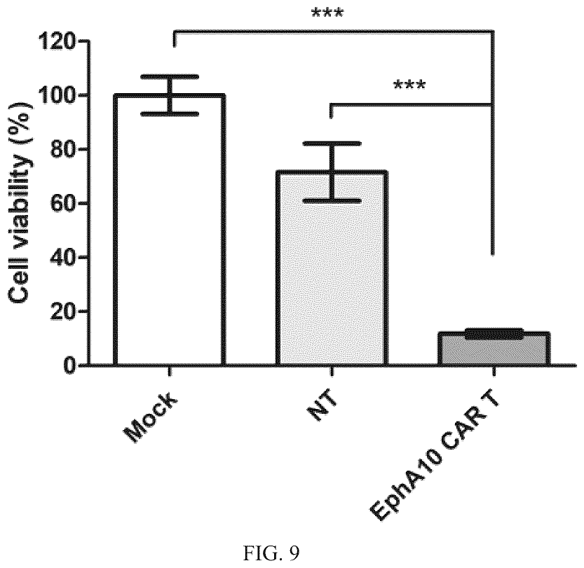

The results are shown in FIG. 9. The treatment of EphA10-CAR-T cells significantly reduced the viability of MDA-MB-231.

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

What is claimed is:

1. An antibody or antigen-binding fragment thereof that is specific to an epitope in ephrin type-A receptor 10 A10 (EphA10); wherein the antibody or antigen-binding fragment thereof comprises complementarity determining regions (CDRs) of a heavy chain variable region and complementarity determining regions of a light chain variable region, wherein the complementarity determining regions of the heavy chain variable region comprise CDRH1, CDRH2 and CDRH3 regions, and the complementarity determining regions of the light chain variable region comprise CDRL1, CDRL2 and CDRL3 regions, and wherein:
the CDRH1 region comprises the amino acid sequence of SEQ ID NO: 1 or a substantially similar sequence thereof; the CDRH2 region comprises the amino acid sequence of SEQ ID NO: 2 or a substantially similar sequence thereof; the CDRH3 region comprises the amino acid sequence of SEQ ID NO: 3 or a substantially similar sequence thereof; and
the CDRL1 region comprises the amino acid sequence of SEQ ID NO: 4 or a substantially similar sequence thereof; the CDRL2 region comprises the amino acid sequence of SEQ ID NO: 5 or a substantially similar sequence thereof; the CDRL3 region comprises the amino acid sequence of SEQ ID NO: 6 or a substantially similar sequence thereof.

2. The antibody or antigen-binding fragment thereof according to claim 1, wherein the antibody or antigen-binding fragment thereof comprises a heavy chain variable region comprising the amino acid sequence of SEQ ID NO: 7 or a substantially similar sequence thereof; and a light chain variable region comprising the amino acid sequence of SEQ ID NO: 8 or a substantially similar sequence thereof.

3. The antibody or antigen-binding fragment thereof according to claim 1, wherein the antibody is a monoclonal antibody, chimeric antibody, humanized antibody or human antibody.

4. The antibody or antigen-binding fragment thereof according to claim 1, which is conjugated with a therapeutic agent.

5. The antibody or antigen-binding fragment thereof according to claim 4, wherein the therapeutic agent is selected from the group consisting of antimetabolites, alkylating agents, alkylating-like agents, DNA minor groove alkylating agents, anthracyclines, antibiotics, calicheamicins, antimitotic agents, topoisomerase inhibitors, HDAC inhibitor, proteasome inhibitors, and radioisotopes.

6. (canceled)

7. (canceled)

8. (canceled)

9. (canceled)

10. The antibody or antigen-binding fragment thereof according to claim 1, comprising
a first antigen binding portion comprising the CDRH1, CDRH2, CDRH3, CDRL1, CDRL2 and CDRL3; and
at least one second antigen binding portion.

11. The antibody or antigen-binding fragment thereof according to claim 10, wherein the second antigen binding portion is an anti-CD3 portion comprising an antibody or antigen-binding fragment thereof that is specific to an epitope in CD3 or an anti-CD16 portion comprising an antibody or antigen-binding fragment thereof that is specific to an epitope in CD16.

12. The antibody or antigen-binding fragment thereof according to claim 11, which comprises
the first antigen binding portion and the anti-CD3 portion;
the first antigen binding portion and the anti-CD16 portion; or
the first antigen binding portion, the anti-CD3 portion and the anti-CD16 portion.

13. The antibody or antigen-binding fragment thereof according to claim 12, which is in an arrangement of [the first antigen binding portion]-[the anti-CD3 portion], [the anti-CD3 portion]-[the first antigen binding portion], [the first antigen binding portion]-[the anti-CD16 portion], [the anti-CD16 portion]-[the first antigen binding portion], [the first antigen binding portion]-[the anti-CD3 portion]-[the anti-CD16 portion], [the first antigen binding portion]-[the anti-CD16 portion]-[the anti-CD3 portion], [the anti-CD3 portion]-[the first antigen binding portion]-[the anti-CD16 portion], [the anti-CD3 portion]-[the anti-CD16 portion]-[the first antigen binding portion], [the anti-CD16 portion]-[the anti-CD3 portion]-[the first antigen binding portion], or [the anti-CD16 portion]-[the first antigen binding portion]-[the anti-CD3 portion] from N-terminus to C-terminus.

14. The antibody or antigen-binding fragment thereof according to claim 10, which further comprises a linker located between any two of the first antigen binding portion and the second antigen binding portion.

15. The antibody or antigen-binding fragment thereof according to claim 10, which further comprises a secretion signal peptide or a protein purification tag.

16. (canceled)

17. (canceled)

18. (canceled)

19. (canceled)

20. (canceled)

21. (canceled)

22. (canceled)

23. A vector encoding the antibody or antigen-binding fragment thereof according to claim 1.

24. (canceled)

25. A genetically engineered cell expressing the antibody or antigen-binding fragment thereof according to claim 1 or a cell differentiated therefrom.

26. The genetically engineered cell or a cell differentiated therefrom according to claim 25, which is an immune cell or a stem cell.

27. (canceled)

28. (canceled)

29. (canceled)

30. (canceled)

31. (canceled)

32. (canceled)

33. (canceled)

34. (canceled)

35. (canceled)

36. A method for inhibiting EphA10-mediated signaling in a subject in need, comprising administering an effective amount of the antibody or antigen-binding fragment thereof according to claim 1 to the subject.

37. A method for treating, prophylactic treating and/or preventing diseases and/or disorders caused by or related to EphA10 activity and/or signaling in a subject afflicted with the diseases and/or disorders, comprising administering an effective amount of the antibody or antigen-binding fragment thereof according to claim 1.

38. The method of claim 16, wherein the diseases and/or disorders are a tumor selected from the group consisting of renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, and melanoma.

39. A method for treating, prophylactic treating and/or preventing diseases and/or disorders caused by or related to EphA10 activity and/or signaling in a subject afflicted with the diseases and/or disorders, comprising administering an effective amount of genetically engineered cell or the cell differentiated therefrom according to claim 13 to the subject.

40. The method according to claim 18, wherein the diseases and/or disorders are a tumor selected from the group consisting of renal cell carcinoma, pancreatic carcinoma, breast cancer, head and neck cancer, prostate cancer, malignant gliomas, osteosarcoma, colorectal cancer, gastric cancer, malignant mesothelioma, multiple myeloma, ovarian cancer, small cell lung cancer, non-small cell lung cancer, synovial sarcoma, thyroid cancer, and melanoma.

41. A method for detecting EphA10 in a sample comprising contacting the sample with the antibody or antigen-binding fragment thereof according to claim 1.

42. A method for neutralizing EphA10 in a subject in need, comprising administering an effective amount of the antibody or antigen-binding fragment thereof according to claim 1 to the subject.

43. (canceled)
